(19) United States
(12) Patent Application Publication
(10) Pub. No.: US 2017/0283769 A1
(43) Pub. Date: Oct. 5, 2017

(54) Modified Cells Evoking Reduced Immunogenic Responses

(71) Applicant: Zhejiang University, Hangzhou (CN)

(72) Inventors: Lei Xiao, Shanghai (CN); Haide Chen, Hangzhou (CN); Chun Cui, Hangzhou (CN); Yang Li, Hangzhou (CN); Di Cui, Hangzhou (CN)

(21) Appl. No.: 15/616,787

(22) Filed: Jun. 7, 2017

Related U.S. Application Data
(63) Continuation-in-part of application No. PCT/CN2015/099322, filed on Dec. 29, 2015.
(60) Provisional application No. 62/104,709, filed on Jan. 17, 2015.

(51) Int. Cl. C12N 5/0735; A61K 35/545

(52) U.S. Cl. CPC C12N 5/0606; A61K 35/545; C12N 2510/00

(57) ABSTRACT

The present disclosure describes composition, methods, and systems associated with a genetically modified stem cell. The modified stem cell includes a reduced amount of Major Histocompatibility Complex II (MHC II) as compared to a corresponding wild-type cell. Further, the modified cell has decreased immunogenicity as compared to the corresponding wild-type cell.

### CROSS REFERENCE TO RELATED PATENT APPLICATIONS

This application is a continuation-in-part of International application number PCT/CN2015/099322, filed Dec. 29, 2015, titled “Modified Cells Evoking Reduced Immunogenic Responses,” which claims the priority benefit to U.S. Provisional Patent Application No. 62104709, filed on Jan. 17, 2015, which is hereby incorporated by reference in its entirety.

### TECHNICAL FIELD

The present disclosure relates to cell therapy. More specifically, the disclosure relates to modified cells evoking reduced immunogenic responses.

### BACKGROUND

Stem cells have the capacity to self-renew and differentiate. Under certain conditions, a stem cell can differentiate into a variety of functional cells. According to the stage of development, stem cells can be divided into two categories: embryonic stem cells (ES cells) and adult stem cells; according to the potential of development, stem cells can be divided into three categories: totipotent stem cells (TSC), pluripotent stem cells and unipotent stem cell. Stem cells are undifferentiated and immature with the ability to regenerate various tissues and organs of a human.

Since the 1990s, several therapy strategies involving stem cells have been attempted to cure diseases. Nevertheless, major obstacles remain to be addressed before clinical applications of stem cell-based cells replacement therapy, such as allograft immune rejection.

### SUMMARY

Embodiments herein relate to a modified cell comprising a reduced amount of Major Histocompatibility Complex II (MHC II) as compared to a corresponding wild-type cell. In these instances, the modified cell has decreased immunogenicity as compared to the corresponding wild-type cell, and the modified cell is a modified stem cell or a cell derived from the modified stem cell.

The embodiments further relate to methods for generating modified stem cells for transplantation. A method may comprise culturing in a culture media modified cells that have a reduced amount of MHC II as compared to corresponding wild-type stem cells. In these instances, the modified stem cells have decreased immunogenicity as compared to the corresponding wild-type stem cells, and the modified cells comprise a modified stem cell or a cell derived from the modified stem cell.

The embodiments further relate to methods for treating a condition. A method may comprise administering to a subject a therapeutically effective amount of modified cells that have a reduced amount of MHC II as compared to corresponding wild-type cells. In these instances, the modified cells have decreased immunogenicity as compared to the corresponding wild-type cells, and the modified cells comprise a modified stem cell or a cell derived from the modified stem cell.

In some embodiments, the modified cell has reduced expression of one or more genes of a biosynthesis or transportation pathway of MHC II as compared to the corresponding wild-type cell.

In some embodiments, the modified cell accumulates a reduced amount of MHC II on the modified cell as compared to the corresponding wild-type cell.

In some embodiments, the modified stem cell has a disruption in an endogenous gene associated with a biosynthesis or transportation pathway of MHC

In some embodiments, the disruption comprises a disruption of MHC class II transactivator (CIITA).

In some embodiments, the disruption results from deletion of at least a portion of CIITA.

In some embodiments, the MHC II comprises Human Leukocyte antigen II (HLA-II).

In some embodiments, the modified stem cell comprises at least one of a totipotent stem cell, a pluripotent stem cell, an embryonic stem cell, an induced pluripotent stem cell, or a multipotent stem cell.

In some embodiments, the modified stem cell comprises a human embryonic stem cell.

In some embodiments, the decreased immunogenicity comprises a decreased level of inflammatory responses induced by the modified cell as compared to the corresponding wild-type cell.

In some embodiments, a karyotype of the modified cell is the same as a karyotype of the corresponding wild-type cell.

In some embodiments, a level of pluripotency of the modified cell is substantially the same as a level of pluripotency of the corresponding wild-type cell.

Some embodiments of the present disclosure relate to a modified cell including a disruption of CIITA. The modified cell is a modified stem cell or a cell derived from the modified stem cell.

In some embodiments, the MHC II is HLA-II.

In some embodiments, the modified stem cell may include at least one of a totipotent stem cell, a pluripotent stem cell, an embryonic stem cell, an induced pluripotent stem cell, or a multipotent stem cell.

In some embodiments, the modified stem cell may include a human embryonic stem cell.

In some embodiments, a karyotype of the modified cell is the same as a karyotype of the corresponding wild-type cell.

In some embodiments, a level of pluripotency of the modified cell is substantially the same as a level of pluripotency of the corresponding wild-type cell.

In some embodiments, the disruption of CIITA may include a disruption of one or more exons of CIITA having the nucleic acid sequence of SEQ ID NO: 46. For example, the one or more exons may include Exon 1 and/or Exon 2 of CIITA.

In some embodiments, the modified cell may include at least one of the nucleic acid sequence of SEQ ID NOs: 47-58.

In some embodiments, the modified cell has decreased immunogenicity as compared to the corresponding wild-type cell.

In some embodiments, the decreased immunogenicity may include a decreased level of inflammatory responses induced by the modified cell as compared to the corresponding wild-type cell.

Some embodiments of the present disclosure relate to a method of reducing T cell responses to the presentation of a cell. The method may include introducing a disruption of CIITA of the cell to obtain a modified cell. The cell is a stem cell or a cell derived from the stem cell. For example, the disruption of CIITA of the cell produces a reduced T cell responses to the presentation of the modified cell as compared to a wild-type cell. In certain embodiments, cells derived from CIITA−/− hESCs may escape from the attack of CD4+ T cells when tissues and/or cells derived from the CIITA−/− hESCs are transplanted into human body. Accordingly, the method provides an effective approach to reduce immune rejection of human ESC-derived allografts.

In some embodiments, the MHC II is HLA-II.

In some embodiments, the stem cell may include at least one of a totipotent stem cell, a pluripotent stem cell, an embryonic stem cell, an induced pluripotent stem cell, or a multipotent stem cell.

In some embodiments, the stem cell may include a human embryonic stem cell.

In some embodiments, the T cell is a CD4+ T-cell.

In some embodiments, a level of pluripotency of the modified cell is substantially the same as a level of pluripotency of the cell.

In some embodiments, the disruption of CIITA gene may include a disruption of one or more exons of CIITA having the nucleic acid sequence of SEQ ID NO: 46.

In some embodiments, the modified cell may include one of the nucleic acid sequence of SEQ ID NOs: 47-58.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 illustrates sequences on exon 2 of human CIITA for target by TALENs (A) and shows the efficiency of generation CIITA-deficient Human embryonic stem cells (hESCs) using TALENs in X1 (B) in accordance with embodiments related to disruption of CIITA in hESCs by TALENs.

FIG. 2 shows immunostaining of pluripotent markers, Nanog, Oct4, SSEA3 and Tra-1-60 in CIITA−/−hESCs in accordance with embodiments related to pluripotency of CIITA targeted hESCs.

FIG. 3 shows HE staining identified three germ layers [mesoderm (left), ectoderm (middle) and endoderm (right)] in teratomas formed from CIITA−/−hESCs. Scale bar 100 μm in accordance with embodiments related to pluripotency of CIITA targeted hESCs.

FIG. 4 shows RT-PCR analysis of differentiated markers expression in CIITA-targeted hESC-derived EBs in accordance with embodiments related to pluripotency of CIITA targeted hESCs.

FIG. 5 illustrates Karyotype analysis of CIITA heterozygous and homozygous hESCs in accordance with embodiments related to pluripotency of CIITA targeted hESCs. Both groups had two samples been analyzed, and no abnormal karyotype was found.

FIG. 6 shows RT-PCR analysis of p2M, CIITA, HLA II (DRA, DQA, DPA) and Ii in hESCs-derived fibroblasts in accordance with embodiments related to CIITA and HLA Class II expression in fibroblasts derived from CIITA targeted hESCs. They were treated with IFN-γ (500 U/ml) for 5 days. The control groups were IFN-γ free. All groups were compared with CIITA+/+IFN-γ free group. Significance was assessed by a t-test. The data are expressed as the mean±SEM. n≧3. ***p<0.001, ** P<0.01.

FIG. 7 shows Western blotting of HLA II and CIITA proteins expression in treated fibroblasts (fibroblasts treated as mentioned above) in accordance with embodiments related to CIITA and HLA Class II expression in fibroblasts derived from CIITA targeted hESCs.

FIG. 8 shows immunostaining analysis of HLA II and CIITA proteins expression in treated fibroblasts (fibroblasts treated as mentioned above) in accordance with embodiments related to CIITA and HLA Class II expression in fibroblasts derived from CIITA targeted hESCs. Scale bar 100 μm.

FIG. 9 shows FACS analysis of HLA I and II proteins expression on the cell surface in treated fibroblasts in accordance with embodiments related to CIITA and HLA Class II expression in fibroblasts derived from CIITA targeted hESCs.

FIG. 10 shows RT-PCR analysis of CD83, CD86, CD11c, DRA, DPA, DQA, CIITA, HLA-E and p2M in DCs (Dendritic cells) derived from CIITA-targeted hESCs in accordance with embodiments related to HLA Class II expression in DCs derived from CIITA targeted hESCs. All groups were compared with CIITA+/+hESCs group. Significance was assessed by a t-test. The data are expressed as the mean±SEM. n≧3. ***p<0.001, **P<0.01.

FIG. 11 illustrates FACS analysis of HLA II expression in DCs, which were defined by the co-expression of CD83 and CD86 in accordance with embodiments related to HLA Class II expression in DCs derived from CIITA targeted hESCs and comparison of the percentage of HLA II+. Significance was assessed by a t-test. The data are expressed as the mean±SEM, n≧3 ***P<0.001, ** P<0.01, *P<0.05.

FIG. 12 shows the morphology of fibroblasts derived from hESCs (A) and Fibroblast-derived from hESCs express Vimentin in accordance with embodiments related to the derivation of Human Fibroblasts from Teratomas (B).

FIG. 13 shows expression of CD86 in fibroblasts derived from hESCs.

FIGS. 14 and 15 show stimulations of various CD86+fibroblasts in response to CD4+T cells. Significance was assessed by a t-test. The data are expressed as the mean±SEM. n≧3. *P<0.05.

### DETAILED DESCRIPTION

Unless defined otherwise, all technical and scientific terms used herein have the same meaning as commonly understood by those of ordinary skill in the art to which the present disclosure belongs. Although any methods and materials similar or equivalent to those described herein can be used in the practice or testing of the present disclosure, preferred methods and materials are described.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence of a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties (e.g., pegylation) or by post-translational modification techniques as would be understood in the art. The term “derivative” also includes within its scope alterations that have been made to a parent sequence including additions or deletions that provide for functionally equivalent molecules.

As used herein, the terms “function” and “functional” and the like refer to a biological, enzymatic, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

“Homology” refers to the percentage number of amino acids that are identical or constitute conservative substitutions. Homology may be determined using sequence comparison programs such as GAP (Deveraux et al., 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

The term “host cell” includes an individual cell or cell culture which can be or has been a recipient of any recombinant vector(s) or isolated polynucleotide of the present disclosure. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the present disclosure. A host cell which comprises a recombinant vector of the present disclosure is a recombinant host cell.

The term “stem cell” refers to biological cells found in multicellular organisms, that can divide (through mitosis) and differentiate into diverse specialized cell types and can self-renew to produce more stem cells. In mammals, there are two broad types of stem cells: embryonic stem cells, which are isolated from the inner cell mass of blastocysts, and adult stem cells, which are found in various tissues.

The term “immunogenicity” of a composition (e.g., a stem cell) refers to the ability of the composition to induce an immune reaction. For example, when a stem cell is transplanted to a subject, the immunogenicity may be attenuated if the stem cell does not contact MHC I and/or MHC II.

By “isolated” is meant a material that is substantially or essentially free from components that normally accompany it in its native state. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The terms “modulating” and “altering” include “increasing” and “enhancing” as well as “decreasing” or “reducing,” typically in a statistically significant or a physiologically significant amount or degree relative to control. In specific embodiments, immunological rejection associated with transplantation of the mammalian stem cell is decreased relative to an unmodified or differently modified stem cell.

An “increased” or “enhanced” amount is typically a “statistically significant” amount, and may include an increase that is 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

By “obtained from” is meant that a sample such as, for example, a polynucleotide or polypeptide is isolated from, or derived from, a particular source, such as the desired organism or a specific tissue within the desired organism. “Obtained from” can also refer to the situation in which a polynucleotide or polypeptide sequence is isolated from, or derived from, a particular organism or tissue within an organism. For example, a polynucleotide sequence encoding a reference polypeptide described herein may be isolated from a variety of prokaryotic or eukaryotic organisms, or from particular tissues or cells within a certain eukaryotic organism.

The term “operably linked” as used herein means placing a gene under the regulatory control of a promoter, which then controls the transcription and optionally the translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the gene from which it is derived. “Constitutive promoters” are typically active, i.e., promote transcription, under most conditions. “Inducible promoters” are typically active only under certain conditions, such as in the presence of a given molecule factor (e.g., IPTG) or a given environmental condition (e.g., particular CO2 concentration, nutrient levels, light, heat). In the absence of that condition, inducible promoters typically do not allow significant or measurable levels of transcriptional activity. For example, inducible promoters may be induced according to temperature, pH, a hormone, a metabolite (e.g., lactose, mannitol, an amino acid), light (e.g., wavelength specific), osmotic potential (e.g., salt-induced), heavy metal, or an antibiotic. Numerous standard inducible promoters will be known to one of skill in the art.

The term “pluripotency” refers to the ability of ES cells that progeny cells of ES cells retain the potential for multilineage differentiation. Maintenance of pluripotency in ES cells appears to involve continual interactions between multiple nuclear factors—this achieving a balance in which some interactions are inhibitory or antagonistic, and others are positive or cooperative; as well as promoting pluripotency, this represses the genes involved in differentiation. Important factors involved in maintaining pluripotency include Oct4, Nanog, and Sox2.

The recitation “polynucleotide” or “nucleic acid” as used herein designates mRNA, RNA, cRNA, rRNA, cDNA or DNA. The term typically refers to a polymeric form of nucleotides of at least 10 bases in length, either ribonucleotides or deoxynucleotides or a modified form of either type of nucleotide. The term includes single and double stranded forms of DNA and RNA.

The terms “polynucleotide variant” and “variant” and the like refer to polynucleotides displaying substantial sequence identity with a reference polynucleotide sequence or polynucleotides that hybridize to a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs that encode these enzymes.

With regard to polynucleotides, the term “exogenous” refers to a polynucleotide sequence that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides, the term “endogenous” or “native” refers to naturally-occurring polynucleotide sequences that may be found in a given wild-type cell or organism. Also, a particular polynucleotide sequence that is isolated from a first organism and transferred to the second organism by molecular biological techniques is typically considered an “exogenous” polynucleotide with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The recitations “mutation” or “deletion,” in relation to the genes associated with MHC II generally refer to those changes or alterations in a stem cell that render the product of that gene non-functional or having reduced function with respect to the synthesis and/or storage of glycogen or biosynthesis of a given lipid. Examples of such changes or alterations include nucleotide substitutions, deletions, or additions to the coding or regulatory sequences of a targeted gene (e.g., CIITA), in whole or in part, which disrupt, eliminate, down-regulate, or significantly reduce the expression of the polypeptide encoded by that gene, whether at the level of transcription or translation, and/or which produce a relatively inactive (e.g., mutated or truncated) or unstable polypeptide. Techniques for producing such alterations or changes, such as by recombination with a vector having a selectable marker, are exemplified herein and known in the molecular biological art. In particular embodiments, one or more alleles of a gene, e.g., two or all alleles, may be mutated or deleted within a stem cell.

The “deletion” of a targeted gene may also be accomplished by targeting the mRNA of that gene, such as by using various antisense technologies (e.g., antisense oligonucleotides and siRNA) known in the art. Accordingly, targeted genes may be considered “non-functional” when the polypeptide or enzyme encoded by that gene is not expressed by the modified photosynthetic microorganism, or is expressed in negligible amounts, such that the modified stem cell produces or accumulates less of the polypeptide or enzyme product (e.g., MHC II) than an unmodified or differently modified stem cell.

In certain aspects, a targeted gene may be rendered “non-functional” by changes or mutations at the nucleotide level that alter the amino acid sequence of the encoded polypeptide, such that a modified polypeptide is expressed, but which has reduced function or activity (e.g., CIITA), whether by modifying that polypeptide's active site, its cellular localization, its stability, or other functional features apparent to a person skilled in the art. Such modifications to the coding sequence of a polypeptide involved in an MHC II biosynthesis or transportation pathway may be accomplished according to known techniques in the art, such as site-directed mutagenesis at the genomic level and/or natural selection (i.e., directed evolution) of a given stem cell.

“Polypeptide,” “polypeptide fragment,” “peptide” and “protein” are used interchangeably herein to refer to a polymer of amino acid residues and to variants and synthetic analogs of the same. Thus, these terms apply to amino acid polymers in which one or more amino acid residues are synthetic non-naturally occurring amino acids, such as a chemical analog of a corresponding naturally occurring amino acid, as well as to naturally occurring amino acid polymers. In certain aspects, polypeptides may include enzymatic polypeptides, or “enzymes,” which typically catalyze various chemical reactions.

“Reducing T cell responses” is intended to mean any means having the effect of abolishing, or otherwise reducing, T cell responses against an antigen, whether by reducing T cell activation, T cell proliferation or by any other inhibitory mechanism, whereby the extent or duration of an immunogenic T cell response is decreased. For example, T cell activation is dependent upon signs transferred through antigen-specific T cells receptor recognition and accessory receptors on the T cell. By introducing a disruption of CIITA of a cell, a level of T cell activation in response to presentation of the modified cell may be reduced about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.), as compared to a level T cell activation associated with a corresponding wild-type cell. T cell activation may be measured based on the expression of the early activation markers CD69, CD62L, CD44, etc.

The recitation polypeptide “variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “reference sequence” generally refers to a nucleic acid coding sequence, or amino acid sequence, to which another sequence is being compared. All polypeptide and polynucleotide sequences described herein are included as references sequences, including those described by name and those described in the Sequence Listing.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given quantity.

“Transformation” refers to the permanent, heritable alteration in a cell resulting from the uptake and incorporation of foreign DNA into the host-cell genome; also, the transfer of an exogenous gene from one organism into the genome of another organism.

The term “wild-type” refers to a gene or gene product that has the characteristics of that gene or gene product when isolated from a naturally-occurring source. A wild-type gene or gene product (e.g., a polypeptide) is that which is most frequently observed in a population and is thus arbitrarily designed in the “normal” or “wild-type” form of the gene.

Embodiments herein relate to a discovery that hypoimmunogenic (e.g., decreased immunogenicity) and compatible stem cells may be obtained by a disruption in an endogenous gene associated with a biosynthesis or transportation pathway of Major Histocompatibility Complex II (MHC II). Cells differentiated from these hypoimmunogenic and compatible stem cells don't have constitutive, and IFN-γ induced HLA II and may attenuate, for example, the effect of T cell-mediated rejection for cell therapy.

The activation of human T cells is based on two signals (TCR-HLA signal and costimulatory signal). HLA molecules are encoded by a large gene family and divided into class I and II. Firstly, professional or non-professional antigen-presenting cells (APCs) degrade proteins into peptides and then load these peptides onto HLA molecules. And then, TCRs of CD4+ and CD8+ T cells recognized the peptides presented by HLA-II and HLA-I, respectively. At the same time, those APCs express a spectrum of costimulatory molecules (e.g., CD80 and CD86), which will interact with complementary molecules of T cells (e.g., CD28 and Cytotoxic T-lymphocyte antigen 4 (CTLA4)).

Both TCR-HLA signal and costimulatory signal are required for activation of T cells. Thus, if inhibiting either of them, T cells would not attack the allografts. It has been proved that hESCs expressing CTLA4-immunoglobulin fusion protein (CTLA4-Ig) and programmed death ligand-1 (PD-L1) can suppress the allogeneic immune response by simultaneously disrupting the costimulatory pathway and activating the T cell inhibitory pathway. This strategy is useful but not generally applicable. For example, T cells derived from hESCs can't be activated with the expression of CTLA4-Ig and PD-L1. So, this approach limits the application of hESCs in clinic immunotherapy, such as hESCs-derived chimeric antigen receptor (CAR)-T, an effective therapy in cancer treatment.

Moreover, unlike mice T cells, activated human T cells express HLA-II. Production of hypoimmunogenic and compatible CAR-T may prevent the rejection mediated by recipients' T cells. Furthermore, DCs may be derived from those hESCs without HLA-II. Though those DCs can't present antigens normally, the CAR technique (CAR-DCs) and artificial HLA-peptide may let these modified cells be more specific and sensitive to the cancers.

HLA-I molecules are found on the surface of each nucleated cells. Constitutive HLA II molecules are expressed mainly on thymic epithelial cells and professional APCs, including DCs, B-lymphocytes, monocytes and macrophages. Under the stress of inflammatory cytokines (e.g., IFN-γ and TNF-α), nonprofessional APCs such as fibroblasts and epithelial cells can also express HLA II molecules, which are known as “induced HLA II.” Each classical HLA-I molecule structurally consists of a polymorphic heavy chain (e.g., HLA-A, HLA-B, and HLA-C), which binds to a same light chain p2M. hESCs with knocked out p2M demonstrated the loss of HLA-I molecules, which endowed hESCs with the capacity of avoiding the CD8+ T cells-mediated rejection. However, no report has demonstrated the generation of hESCs with the ability to differentiate into cells without constitutive, and IFN-y induced HLA-II.

The embodiments further related to a modified cell comprising a reduced amount of MHC II as compared to a corresponding wild-type cell. In these instances, the modified cell has decreased immunogenicity as compared to the corresponding wild-type cell, and the modified cell is a modified stem cell or a cell derived from the modified stem cell.

The embodiments further relate to methods for generating modified stem cells for transplantation. A method may comprise culturing in a culture media modified cells that have a reduced amount of MHC II as compared to corresponding wild-type stem cells. In these instances, the modified stem cells have decreased immunogenicity as compared to the corresponding wild-type stem cells, and the modified cells comprise a modified stem cell or a cell derived from the modified stem cell. For example, the disruption of CIITA of the cell produces a reduced T cell responses to the presentation of the modified cell as compared to a wild-type cell. In certain embodiments, cells derived from CIITA−/− hESCs may escape from the attack of CD4+ T cells when tissues and/or cells derived from the CIITA−/− hESCs are transplanted into human body. Accordingly, the method provides an effective approach to reduce immune rejection of hESCs derived allografts.

The embodiments further relate to methods for treating a condition. A method may comprise administering to a subject a therapeutically effective amount of modified cells that have a reduced amount of MHC II as compared to corresponding wild-type cells. In these instances, the modified cells have decreased immunogenicity as compared to the corresponding wild-type cells, and the modified cells comprise a modified stem cell or a cell derived from the modified stem cell.

In some embodiments, the condition may comprise at least one of Parkinson's disease, Huntington's disease, Alzheimer's disease, amyotrophic lateral sclerosis, spinal muscular atrophy, a neurodegenerative disease psychosis, as well as spinal cord injury, stroke, burns, heart disease, liver disease, diabetes, blood cancer, organ transplantation defects and damage regeneration.

In some embodiments, the modified cell has reduced expression of one or more genes of a biosynthesis or transportation pathway of MHC II as compared to the corresponding wild-type cell.

In some embodiments, the modified cell accumulates a reduced amount of MHC II on the modified cell as compared to the corresponding wild-type cell.

In some embodiments, the modified stem cell has a disruption in an endogenous gene (e.g., RFX factors (RFXAP, RFXS, RFXANK) and CIITA) associated with a biosynthesis or transportation pathway of MHC II. In certain embodiments, the disruption comprises a disruption of CIITA. For example, the disruption results from deletion of at least a portion of CIITA.

HLA II genes are regulated by a same regulatory complex consists of three RFX factors (RFXAP, RFXS, RFXANK) and CIITA. This complex regulates not only the genes encoding classical HLA II molecules (HLA-DP, HLA-DQ, and HLA-DR) but also the genes encoding accessory proteins that are required for intracellular transportation and peptide loading of HLA II molecules, including the non-classical HLA II molecules (invariant chain (Ii), HLA-DM and HLA-DO). In some cases, tumor cells and virus-infected cells will escape CD4+ T cells-mediated immune rejection via silencing the HLA II. Here using TALENs technique, HLA-II molecules of hESCs are disrupted by knocking out CIITA—the master regulator of HLA II molecules. The main function of CIITA is HLA II regulation, so they have almost same cellular distribution. CIITA does not bind DNA directly but interacts with other elements consisting of cyclic AMP response element-binding protein (CREB), nuclear factor Y complex (NF-Y) and RFX factors (RFXS, RFXANK, RFXAP). Patients without functional CIITA are suffering from bare lymphocyte syndrome (BLS), which is characterized by the lack of expression of HLA II in tissue cells. CIITA−/− mice are also impaired in MHC class II-mediated allogeneic responses.

CIITA has four promoters, and they can regulate HLA II expression in a tissue-specific manner. To target CIITA thoroughly, TALENs are designed in the communal exons (exon 2 and 3) of all transcripts. hESCs don't express HLA II and CIITA in vitro even during the embryoid bodies (EBs) differentiation or IFN-γ induction. The constitutive and induced HLA II molecules on hESCs-derived DCs and fibroblasts are checked, respectively. The deletion of CIITA may decrease the constitutive and induced expression of HLA II molecules dramatically.

In some embodiments, the MHC II comprises HLA-II. In these instances, the modified stem cell comprises a human embryonic stem cell.

In some embodiments, the modified stem cell comprises at least one of a totipotent stem cell, a pluripotent stem cell, an embryonic stem cell, an induced pluripotent stem cell, or a multipotent stem cell.

In some embodiments, the decreased immunogenicity comprises a decreased level of inflammatory responses induced by the modified cell as compared to the corresponding wild-type cell, for example, when the modified cell is transplanted to a subject.

In some embodiments, a karyotype of the modified cell is the same as a karyotype of the corresponding wild-type cell.

In some embodiments, a level of pluripotency of the modified cell is substantially the same as a level of pluripotency of the corresponding wild-type cell. In these instances, cells differentiated (e.g., fibroblasts and epithelial cells) from the modified stem cell don't have constitutive, and IFN-γ induced HLA-II.

Some embodiments of the present disclosure relate to a modified cell including a disruption of CIITA. The modified cell is a modified stem cell or a cell derived from the modified stem cell.

In some embodiments, the MHC II is HLA-II.

In some embodiments, the modified stem cell may include at least one of a totipotent stem cell, a pluripotent stem cell, an embryonic stem cell, an induced pluripotent stem cell, or a multipotent stem cell.

In some embodiments, the modified stem cell may include a human embryonic stem cell.

In some embodiments, a karyotype of the modified cell is the same as a karyotype of the corresponding wild-type cell.

In some embodiments, a level of pluripotency of the modified cell is substantially the same as a level of pluripotency of the corresponding wild-type cell.

In some embodiments, the disruption of CIITA may include a disruption of one or more exons of CIITA having the nucleic acid sequence of SEQ ID NO: 46.

In some embodiments, the modified cell may include at least one of the nucleic acid sequence of SEQ ID NOs: 47-58.

In some embodiments, the modified cell has decreased immunogenicity as compared to the corresponding wild-type cell.

In some embodiments, the decreased immunogenicity may include a decreased level of inflammatory responses induced by the modified cell as compared to the corresponding wild-type cell.

Some embodiments of the present disclosure relate to a method of reducing T cell responses to the presentation of a cell. The method may include introducing a disruption of CIITA of the cell to obtain a modified cell. The cell is a stem cell or a cell derived from the stem cell.

In some embodiments, the MHC II is HLA-II.

In some embodiments, the stem cell may include at least one of a totipotent stem cell, a pluripotent stem cell, an embryonic stem cell, an induced pluripotent stem cell, or a multipotent stem cell.

In some embodiments, the stem cell may include a human embryonic stem cell.

In some embodiments, the T cell is a CD4+ T-cell.

In some embodiments, a level of pluripotency of the modified cell is substantially the same as a level of pluripotency of the cell.

In some embodiments, the disruption of CIITA may include a disruption of one or more exons of CIITA having the nucleic acid sequence of SEQ ID NO: 46.

In some embodiments, the modified cell may include one of the nucleic acid sequence of SEQ ID NOs: 47-58.

The various embodiments described above can be combined to provide further embodiments. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### EXAMPLES

Disruption of CIITA in hESCs by TALENs

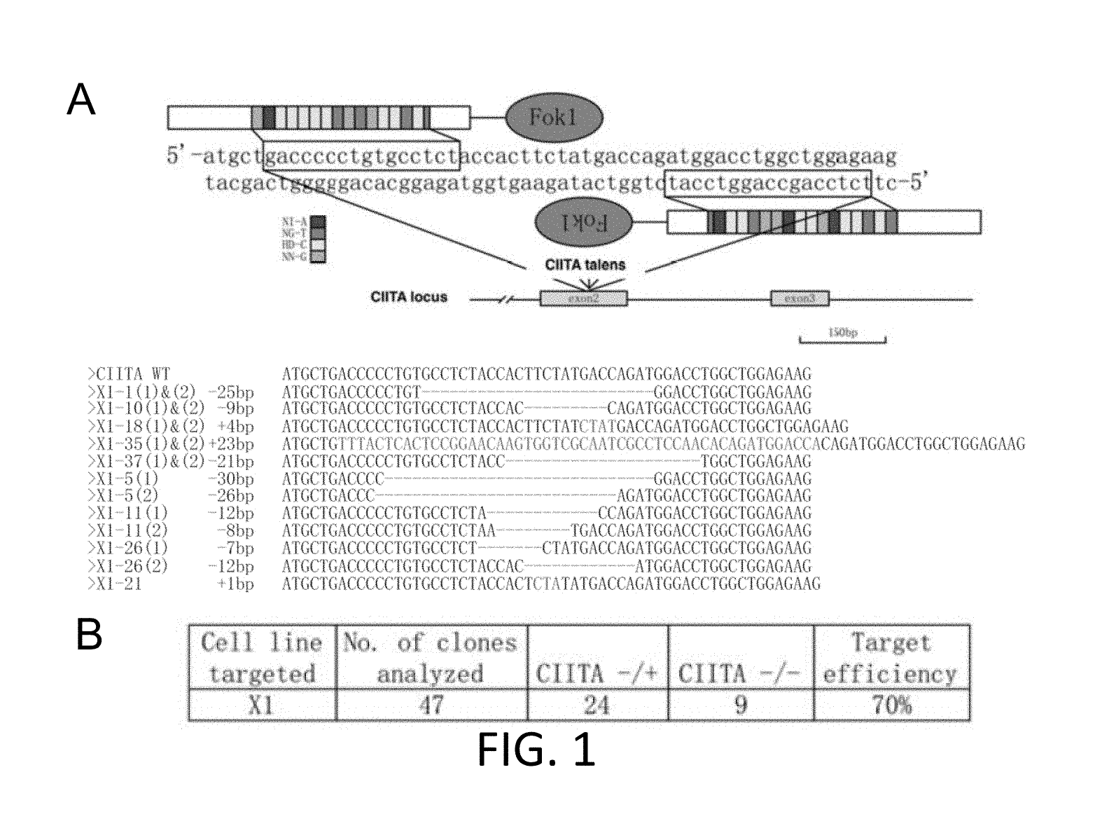

Knockout CIITA in hESCs were performed using TALENs. TALENs of CIITA were designed for exon 2 and exon 3 targeting. The most efficient TALEN pairs (2L2 and 2R2) were selected from the 293T test and were used to target CIITA in X1 hESCs (FIG. 1). Both heterozygous (CIITA+/−) and homozygous (CIITA−/−) hESCs were obtained in one targeting round with the efficiency of 70% (FIG. 1 and Table 1).

Data are represented as the mean±SEM. The data were analyzed statistically using GraphPad Prism 5.1 (GraphPad Software Inc., USA). Performance variants were analyzed by the unpaired Student's T test. Statements of significance were based on *P<0.05 unless otherwise stated.

hESCs Culturing

hESCs were cultured with the Irradiated CF1 feeder cells (3×104 cells/cm2) in the T25 flasks (Corning) coated with Matrigel (Becton-Dickinson). hESCs were maintained in DMEM/F12 (Invitrogen) supplemented with 20% knockout serum replacement (Invitrogen), 4 ng/mL basic fibroblast growth factor (bFGF; Invitrogen), 2 mmol/L I-glutamine (Invitrogen), 1% nonessential amino acids (Invitrogen) and 0.1 mmol/L β-mercaptoethanol (Sigma-Aldrich). hESCs were passaged approximately once a week. Collagenase IV was used to dissociate the cells from the feeders as cell clumps, which were dissociated to an appropriate size before being passaged onto newly prepared feeder cells.

### TALENs Efficiency Detection

TALENs for CIITA were designed to target exon2 (2L1: gctgaccccctgtgcct (SEQ ID NO: 1); 2L2: gaccccctgtgcctct (SEQ ID NO: 2); 2R1: ctccagccaggtccatct (SEQ ID NO: 3); 2R2: tctccagccaggtccat (SEQ ID NO: 4)) and exon3 (3L1: tcagcaggctgttgt (SEQ ID NO: 5); 3L2: tcagcaggctgttgtgt (SEQ ID NO: 6); 3R1: ccctggtctcttcat (SEQ ID NO: 7); 3R2: aagcctccctggtctt (SEQ ID NO: 8); 3R3: aagcctccctggtct (SEQ ID NO: 9)). The TALENs were constructed with FastTALE TALEN Assembly Kit (Sidansai), and their activities were confirmed in 293T cells as the previous description. The constructed TALENs were transfected into 293T cells and selected with 2 μg/ml puromycin (Sigma). The genomic DNA of 293T cells was harvested after selection. Then, PCR and sequencing were performed to examine the efficiency of the TALENs.

Generation of CIITA-Deficient hESCs

To prepare the cells for transfection, harvested hESCs were plated in six-well plates coated with Matrigel in mTeSR™ 1 medium (Stemcell Technologies). On the following day, the most efficient TALENs (2L2 and 2R2) plasmids and EGFP-Puro plasmid (Sidansai) (1:1:1) were transfected into hESCs by the FuGENE HD transfection reagent (Promega). The FuGENE HD Transfection Reagent/plasmids/Opti-MEM (Life Technologies) mixture (15 ul/6 ug/300 ul) was incubated for 15 min at room temperature, and then the mixture was added to the cell culture. Puromycin was added into media two days later. After selection with 0.5 μg/ml puromycin, the survival colonies were dissociated into single cells using TrypLE (Invitrogen) and seeded onto CF1-coated plates at a density of 500 cells/cm2. Two weeks after passaging, the colonies derived from the single cells were transferred into freshly CF1-coated wells, and in parallel, a direct cell PCR kit was used to identify the mutants.

The Pluripotency of CIITA Targeted hESCs

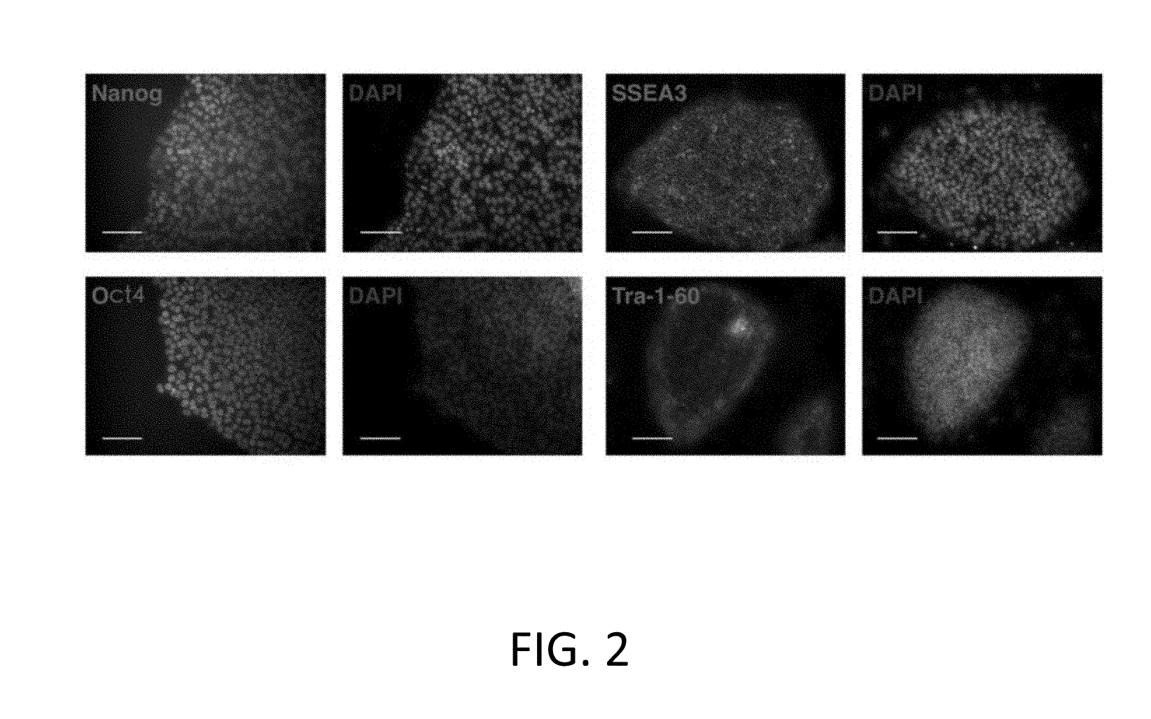

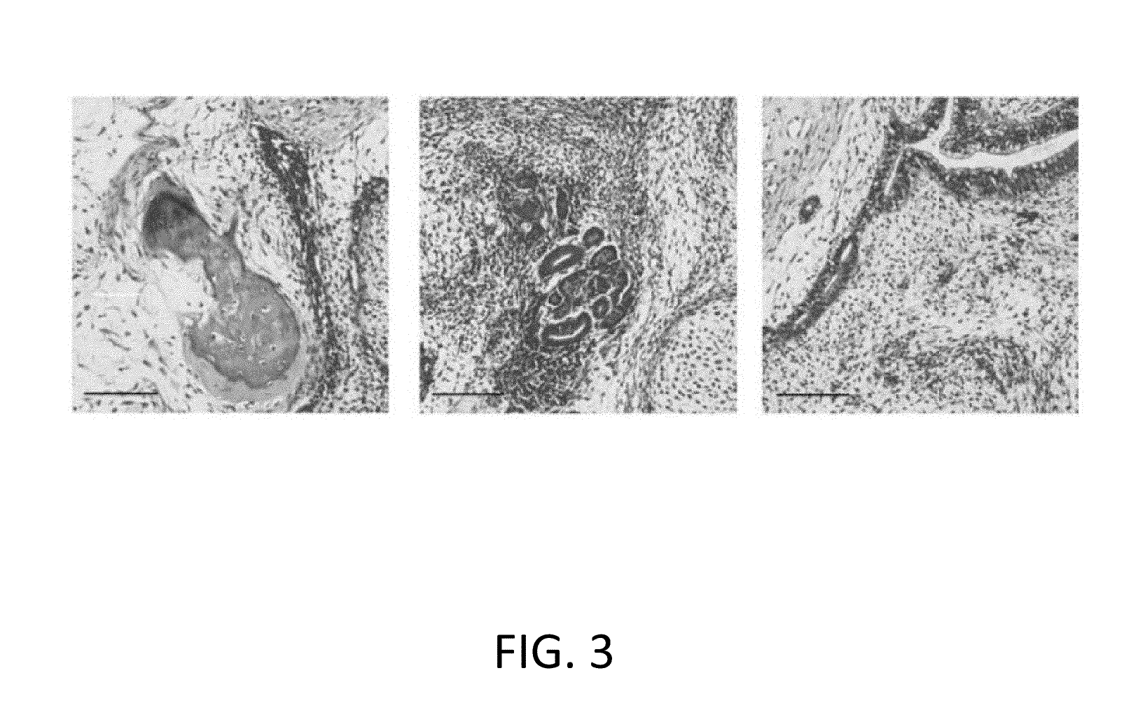

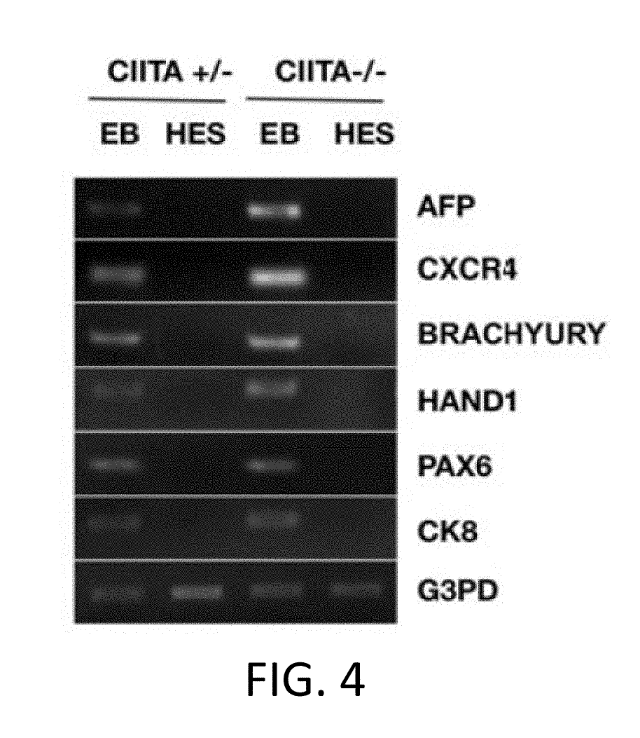

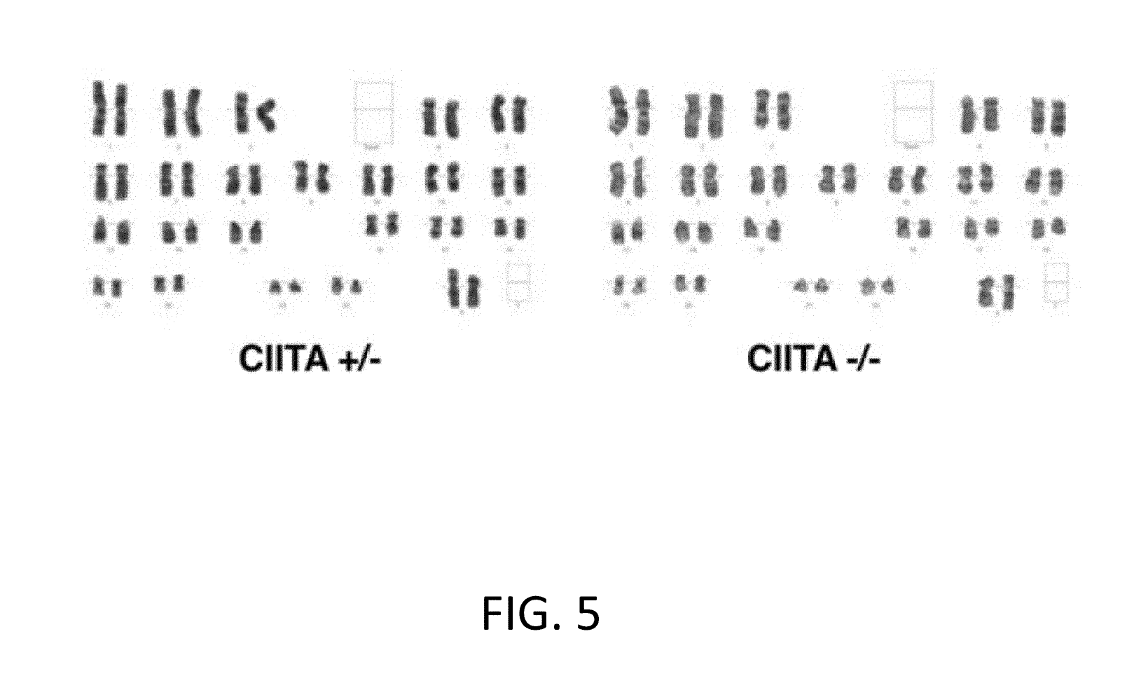

The pluripotency of hESCs is necessary for its application in cells replacement therapy. So, the pluripotency in established CIITA-targeted hESCs was investigated. Immunostaining showed that CIITA−/− hESCs were positive for Oct4, Nanog, Tra-1-60 and SSEA3 (FIG. 2). When CIITA−/− hESCs were injected into non-obese diabetic/severe combined immune-deficient (NOD/SCID) mice, teratomas formed after 2 months, and tissues derived from three germ layers were observed in hematoxylin-eosin (HE) staining section (FIG. 3). EBs derived from CIITA−/− hESCs were performed RT-PCR to show the expression of three germ layers markers (FIG. 4). Moreover, both CIITA−/− and CIITA+/− hESCs had normal karyotypes (FIG. 5). The normal karyotype lines were used in the following experiments. Thus, the targeted hESCs did not show any difference in pluripotency and karyotypes. Primers for RT-PCT and related sequences were listed in table 1. Several cell lines (x1-1-35, x1-1-21, x1-1-18 and CIITA WT) were used for differentiation and further assays.

TABLE 1

| Gene name | | Sequence | SEQ ID NO |
|---|---|---|---|
| CD8 | F | tacacggtctcctgggtcaag | SEQ ID NO: 10 |
| 3 | R | gtctcttctttacgctgtgcag | SEQ ID NO: 11 |
| CD8 | F | tctcaagataatgtcacagaactg | SEQ ID NO: 12 |
| 6 | R | tctgttcactctcttccctctc | SEQ ID NO: 13 |
| CD1 | F | aatgtcgtgcaccgattgttcc | SEQ ID NO: 14 |
| 1c | R | actgcttgtggtctccgtacc | SEQ ID NO: 15 |
| DRA | F | gccgagttctatctgaatcctg | SEQ ID NO: 16 |
| | R | cgttctgctgcattgcttttgc | SEQ ID NO: 17 |
| DPA | F | tatcagagctgtgatcttgagag | SEQ ID NO: 18 |
| | R | atcgttggtggcctgagtgtg | SEQ ID NO: 19 |
| DQA | F | acgctacaactctaccgctgc | SEQ ID NO: 20 |
| | R | atgatgaagacagtgcccacc | SEQ ID NO: 21 |
| Ii | F | tgcacctgctccagaatgctg | SEQ ID NO: 22 |
| | R | ccagttccagtgactctttcg | SEQ ID NO: 23 |
| CIIT | F | gctgggattcctacacaatgc | SEQ ID NO: 24 |
| A | R | gggttctgagtagagctcaatc | SEQ ID NO: 25 |
| HLA- | F | tctcacagcttgtaaagcctgagac | SEQ ID NO: 26 |
| E | R | aggatggtttcgatctcctgacct | SEQ ID NO: 27 |
| B2 | F | atgtgtctgggtttcatccatccg | SEQ ID NO: 28 |
| M | R | tcttcaaacctccatgatgctgct | SEQ ID NO: 29 |
| Vimen- | F | aagaacacccgcaccaacgag | SEQ ID NO: 30 |
| tin | R | ctcctggatttcctcttcgtg | SEQ ID NO: 31 |
| Afp | F | gaatgctgcaaactgaccacgctggaac | SEQ ID NO: 32 |
| | R | tggcattcaagagggttttcagtctgga | SEQ ID NO: 33 |
| Cxcr | F | tcctctttgtcatcacgcttccct | SEQ ID NO: 34 |
| 4 | R | tccagacgccaacatagaccacc | SEQ ID NO: 35 |
| Brachy- | F | gccctctccctcccctccacgcacag | SEQ ID NO: 36 |
| ury | R | cggcgccgttgctcacagaccacagg | SEQ ID NO: 37 |
| Han | F | tgcctgagaaagagaaccag | SEQ ID NO: 38 |
| d1 | R | atggcaggatgaacaaacac | SEQ ID NO: 39 |
| Pax | F | taccaaccaattccacaacccacc | SEQ ID NO: 40 |
| 6 | R | atcataactccgcccattcaccg | SEQ ID NO: 41 |
| Ck8 | F | cctggaagggctgaccgacgagatcaa | SEQ ID NO: 42 |
| | R | cttcccagccaggctctgcagctcc | SEQ ID NO: 43 |
| G3P | F | aggtcggagtcaacggatttgg | SEQ ID NO: 44 |
| D | R | aggctgttgtcatacttctcatgg | SEQ ID NO: 45 |

| | | |
|---|---|---|
| CIITA WT | atgctgaccccctgtgcctctaccacttctatgaccagatggacctgg | SEQ ID NO: 46 |
| | ctggagaag | |
| X1-1-1 | atgctgaccccctgt ggacctggctggagaag | SEQ ID NO: 47 |
| X1-1-10 | atgctgaccccctgtgcctctaccaccagatggacctggctggagaa | SEQ ID NO: 48 |
| | g | |
| X1-1-18 | atgctgaccccctgtgcctctaccacttctatctatgaccagatggacc | SEQ ID NO: 49 |
| | tggctggagaag | |
| X1-135 | atgctgtttactcactccggaacaagtggtcgcaatcgcctccaacac | SEQ ID NO: 50 |
| | agatggaccacagatggacctggctggagaag | |
| X1-1-37 | atgctgaccccctgtgcctctacctggctggagaag | SEQ ID NO: 51 |
| X1-1-5(1) | atgctgaccccggacctggctggagaag | SEQ ID NO: 52 |
| X1-1-5(2) | atgctgacccagatggacctggctggagaag | SEQ ID NO: 53 |
| X1-1-11)1) | atgctgaccccctgtgcctctaccagatggacctggctggagaag | SEQ ID NO: 54 |
| X1-1-11(2) | atgctgaccccctgtgcctctaatgaccagatggacctggctggaga | SEQ ID NO: 55 |
| | ag | |
| X1-1-26(1) | atgctgaccccctgtgcctctctatgaccagatggacctggctggaga | SEQ ID NO: 56 |
| | ag | |
| X1-1-26(2) | atgctgaccccctgtgcctctaccacatggacctggctggagaag | SEQ ID NO: 57 |
| X1-1-21 | atgctgaccccctgtgcctctaccactctatatgaccagatggacctg | SEQ ID NO: 58 |
| | gctggagaag | |

CIITA and HLA II Expression in Defined Cells Derived from CIITA Targeted hESCs

For cell replacement therapy, differentiated cells are transplanted into the subject body directly. Some tissue cells (e.g., professional APCs and thymic epithelial cells) have constitutive expression of HLA II molecules and some other tissue cells (e.g., fibroblasts and epithelial cells) have induced expression of HLA-II molecules. In order to ensure the functional disruption of HLA II, both kinds of HLA II expression in defined types of cells derived from CIITA targeted hESCs were investigated.

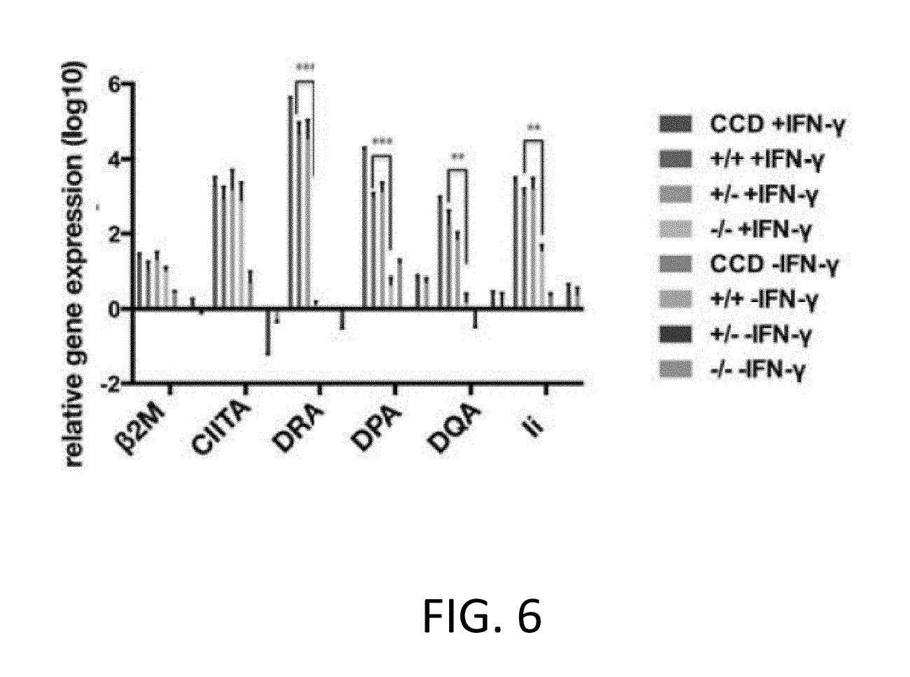

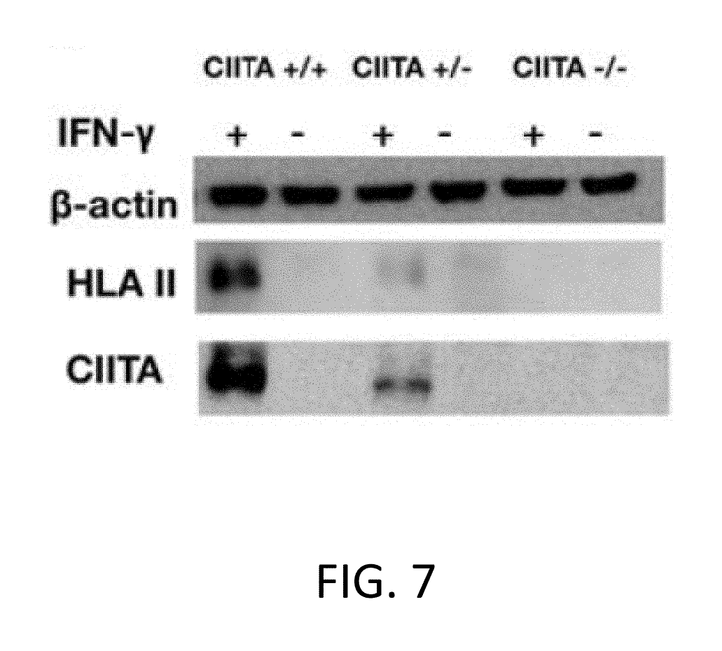

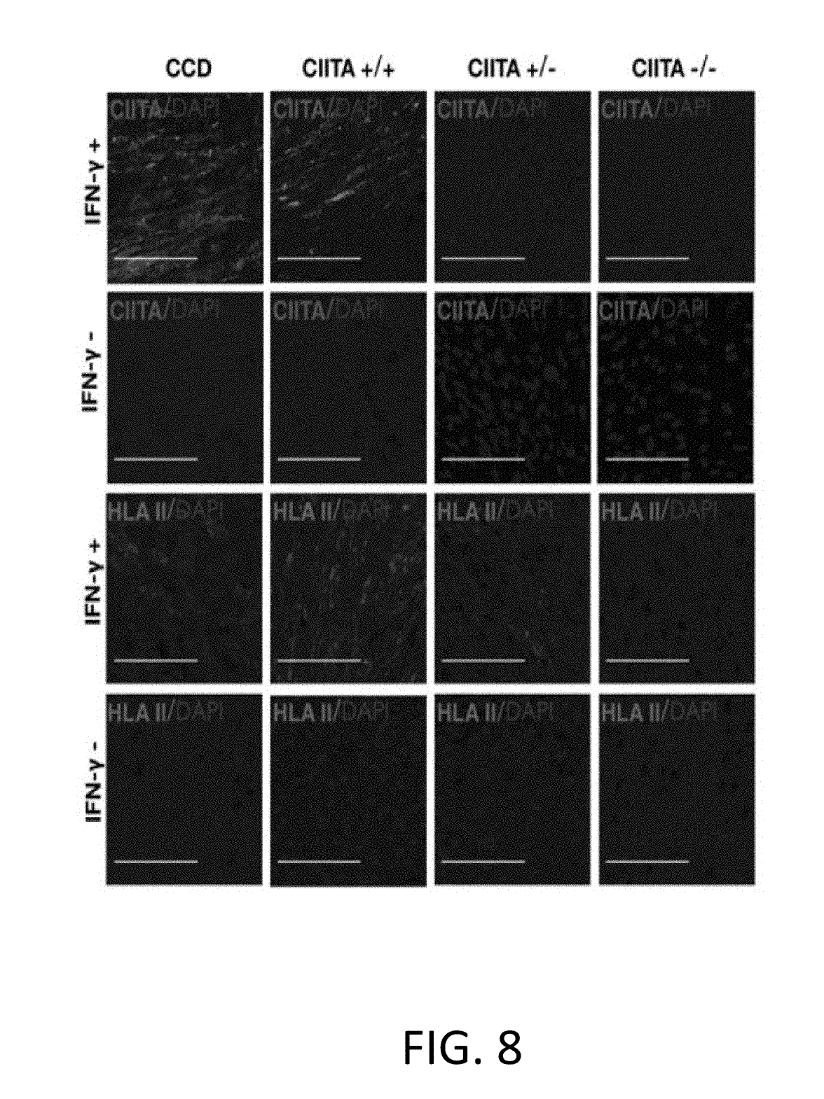

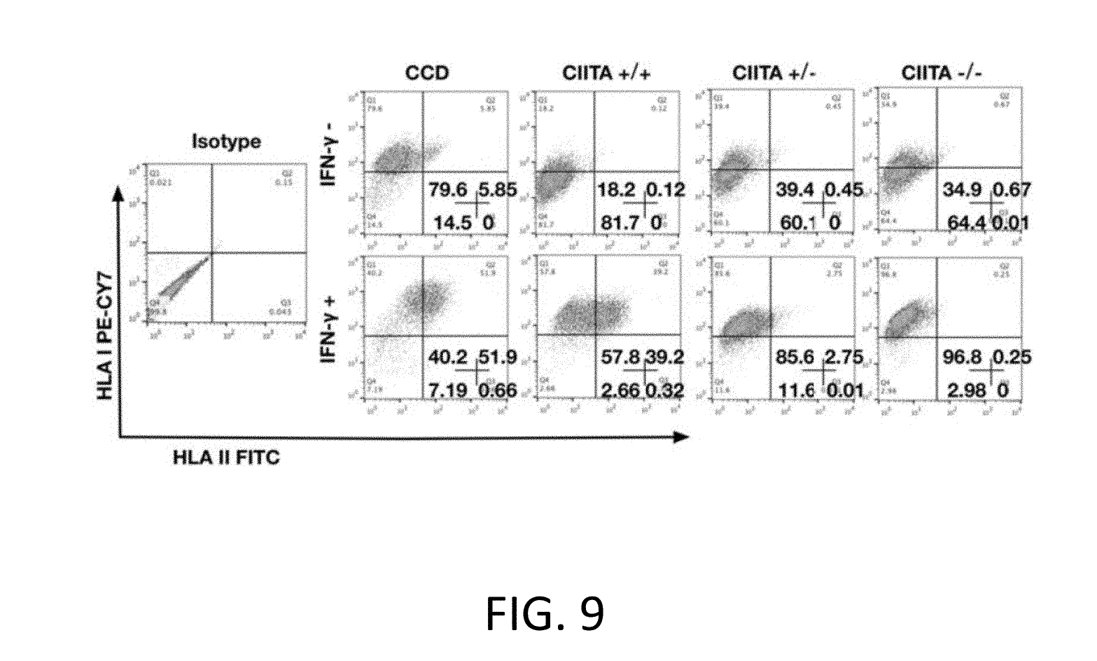

Firstly, IFN-γ inducible HLA II on hESCs-derived fibroblasts with 5 days' treatment of 500 U IFN-γ was checked. CCD-1079SK (CCD) cell line, a human fibroblast cell line, was used as a positive control. IFN-γ induction can increase the expression of p2M in tissue cells. Without IFN-γ treatment, all cells showed low-level expression of HLA II DRA, DPA, DQA, Ii) and β2M. With IFN-γ treatment, β2M and CIITA mRNA increased in all groups as reported (FIG. 6). CIITA targeting did not affect the transcription of CIITA. CIITA +/+ and CIITA+/− fibroblasts increased mRNA expression of HLA II (DRA, DPA, DQA, Ii) as CCD cells did after IFN-γ treatment (FIG. 6). However, IFN-γ treated CIITA−/− fibroblasts didn't increase mRNA expression of HLA II (DRA, DPA, DQA, Ii) clearly (FIG. 6). It suggested that CIITA mRNA detected in IFN-γ treated CIITA−/− fibroblasts was dysfunctional and couldn't be translated into a functional protein to regulate the expression of HLA II (FIG. 6). It was proved by the following Western blotting and Immunochemistry data (FIGS. 7, 8). It also indicated that CIITA+/− fibroblasts had a low-level increase of CIITA and HLA II protein lagged behind the increase of mRNAs (FIGS. 7, 8). FACS analysis of all groups demonstrated that few cells expressed HLA II on cell surface without IFN-γ induction. After IFN-γ induction, CCD and CIITA+/+ fibroblasts increased expression of HLA I and II dramatically. However, neither CIITA+/− nor CIITA−/− increased expression of HLA II clearly (FIG. 9).

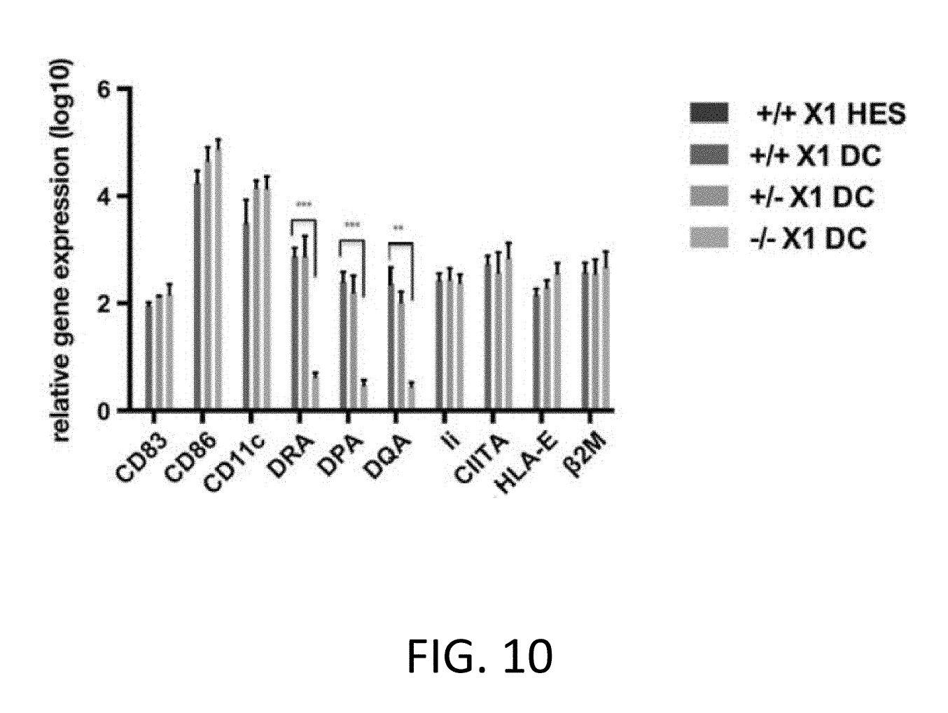

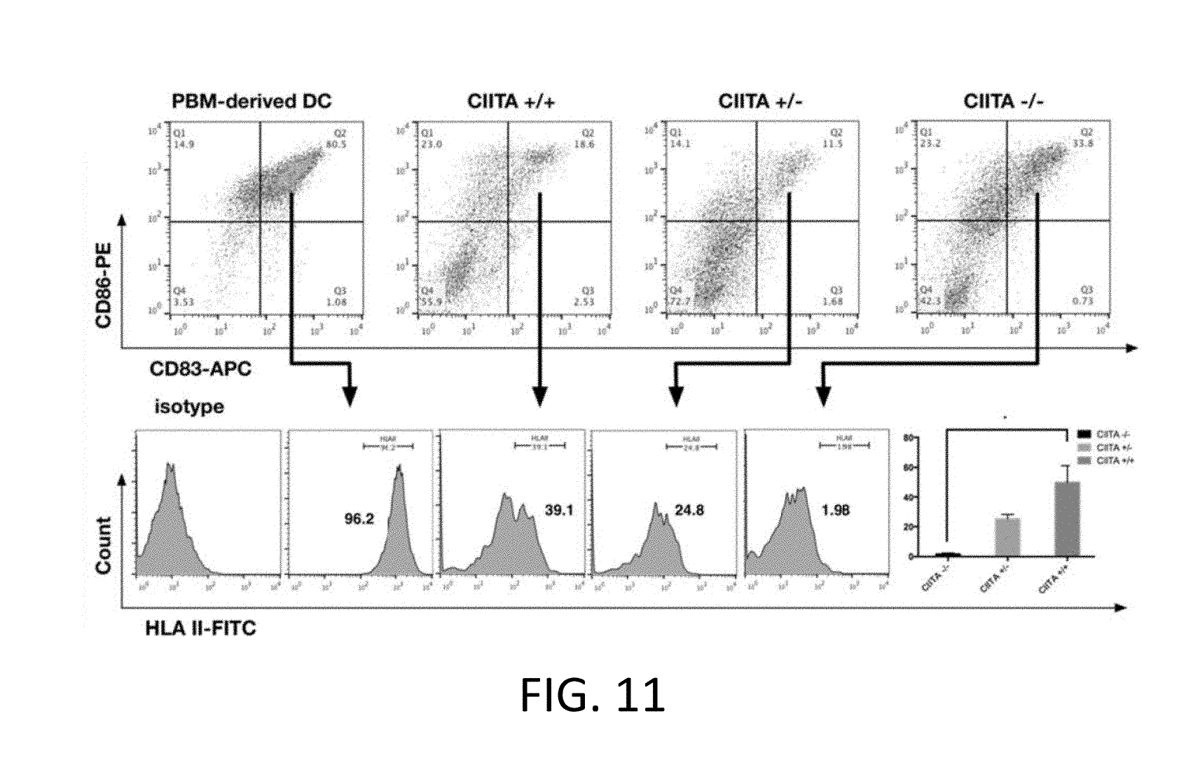

Secondly, DCs were derived from hESCs to test constitutive HLA II expression. Focused on clinical using, a protocol with definitive chemical composition media without serum, feeder or other animal products was chosen. DCs derived from hESCs expressed CD83 and CD86 (FIG. 10, 10). Compared with CIITA+/+ and CIITA+/− DCs, lower level of classical HLA II molecules (DRA, DQA and DPA) mRNA expression was found in CIITA−/− DCs dramatically (FIG. 10). However, non-classical HLA II (Ii) did not show any difference in mRNA expression among them (FIG. 10). Both classical HLA II (HLA-DP, HLA-DQ, and HLA-DR) and non-classical HLA II (HLA-DM, HLA-DO, Ii) have a same specific regulatory module, which can be recognized by RFX-CIITA complex. It has been shown that HLA-DR expression was completely dependent on CIITA, which may cause the residual expression of other HLA II molecules in CIITA-targeted cells (FIGS. 6, 10). Accordingly, Ii had different trends between DCs and fibroblasts, and it indicated a different regulatory pathway of Ii independent of CIITA. The expression of Ii in IFN-γ induced fibroblasts, and DCs may both depend mainly on CIITA, while DCs differentiation last such a long time to activate the substituted regulation pathway without CIITA. It appeared that Ii is encoding accessory proteins, which is required for peptide loading of HLA II molecules and can't rescue the loss of DRA, DPA and DQA on the cell surface (FIGS. 9, 11).

DCs were defined with CD83 and CD86 and compared the percentage of HLA II+ cells in CD83+CD86+DCs. PBM-derived DCs showed the high overlap of those three markers (FIG. 11). CIITA−/− DCs had 1.9% HLA II+ cells while CIITA+/+ and CIITA+/− DCs had a higher percentage of HLA II+ cells, 39.1 and 24.8% respectively.

Teratomas Formation and Derivation of Human Fibroblasts from Teratomas

hESCs were injected intramuscularly into 6-8 weeks NOD/SCID mice (approximately 5×106 cells per site). After about 2 months, the tumors were processed for hematoxylin-eosin (HE) staining.

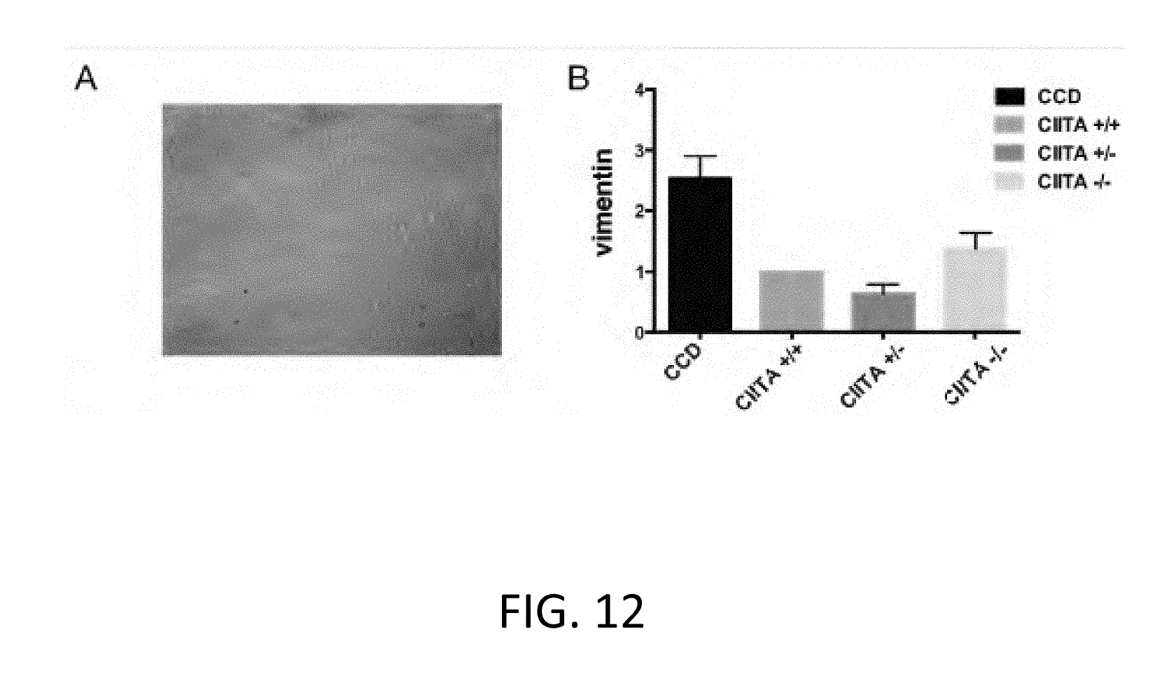

The fibroblast-like cells were also derived from teratomas. Teratomas were cut into pieces with scissors and cultured in DMEM supplemented with 10% serum, 1% Pen-Strep, and 50 uM β-nnecaptoethanol. After several passages, the adherent cells become homogenous and fibroblast-like cells. Cell morphological observation and RT-PCR were performed (FIG. 12). Ten cell lines were established (3 for +/+; 3 for +/−; 4 for −/−). And some mesenchymal stem cells markers in established cells lines (n>3) were analyzed. CCD and mesenchymal stem cells (MSC) were used as a control. Those cell lines were more like fibroblasts. It showed that this method was reproducible in these experiments.

Derivation of Human DCs from hESCs

The differentiation of DCs from hESCs was induced by step-wise growth factors induction in suspension culture of EBs as previously reported. In the first 5 days, hESCs showed mesoderm specification in the X-VIVO™ 15 medium (Lonza) supplemented with 1 mm sodium pyruvate, 1× non-essential amino acids, 2 mm 1-glutamine, 50 mm 2-mercaptoethanol and the four growth factors, including recombinant human bone morphogenetic protein-4 (rhBMP-4; BD), recombinant human vascular endothelial growth factor (rhVEGF; R&D), recombinant human granulocyte-macrophage-colony-stimulating factor (rhGM-CSF; R&D) and recombinant human stem cell factor (rhSCF; R&D). From day 6 to day 10, rhBMP-4 was removed, and the cells became hematopoietic stem cells (HSCs). From day 11 to day 15, rhVEGF was removed, and HSCs turned into common myeloid progenitors (CMP). From day 16 to day 20, rhSCF was removed, and monocyte-like cells appeared and accumulated gradually as DC precursors. DC precursors will become immature DCs (iDCs) with the treatment of rhGM-CSF and recombinant human Interleukin 4 (rhlL-4; R&D) in the next 4-6 days. The maturation of DCs needed further incubation for 1-2 days with the mixed factors, including rhGM-CSF, recombinant human Interleukin-1 beta (rhIL1-β; R&D), recombinant human Interferon gamma (rhIFN-γ; R&D), Prostaglandin E2 (PGE-2; Sigma) and recombinant human Tumor necrosis factor alpha (rhINF-α; R&D).

CD4+T Cells Stimulation with Fibroblasts Derived from Human Pluripotent Stem Cell

Isolation PBMC (Peripheral Blood Mononuclear Cell) was performed from volunteer's peripheral blood. 20 ml blood was obtained from the volunteers. The blood was diluted with an equal amount of PBS (phosphate-buffered saline), and layer blood on top of ficoll. Centrifugation was performed at 400 g for 20 minutes at room temperature with brake off. The mononuclear cell layer and erythrocyte/granulocyte pellet were collected respectively. Both cells were washed twice with PBS for use.

Isolation of CD4+T cells from PBMC were performed. RBCs and Mononuclear Cell were mixed at a ratio of 100 RBCs per nucleated cell. Centrifugation was performed at 1500 rpm for 15 minutes. Cells were resuspended in 1 ml PBS and added to 50 μl/ml RosetteSep™ Human CD4+ T Cell Enrichment Cocktail. The solutions were mixed and incubate at room temperature for 20 minutes. Samples were diluted with 2% PBS and mixed. Diluted samples were added to the tube containing the density gradient medium. Centrifugation was performed with 2000 rpm for 20 minutes, and brake-off was observed. Enriched cell layer was collected with a pipette and transferred to a new tube. The cell layer was washed with 2% PBS and centrifuged 300 g 5 minutes twice. The cells were re-suspended in X-VIVO15, and the cells were counted for further processing.

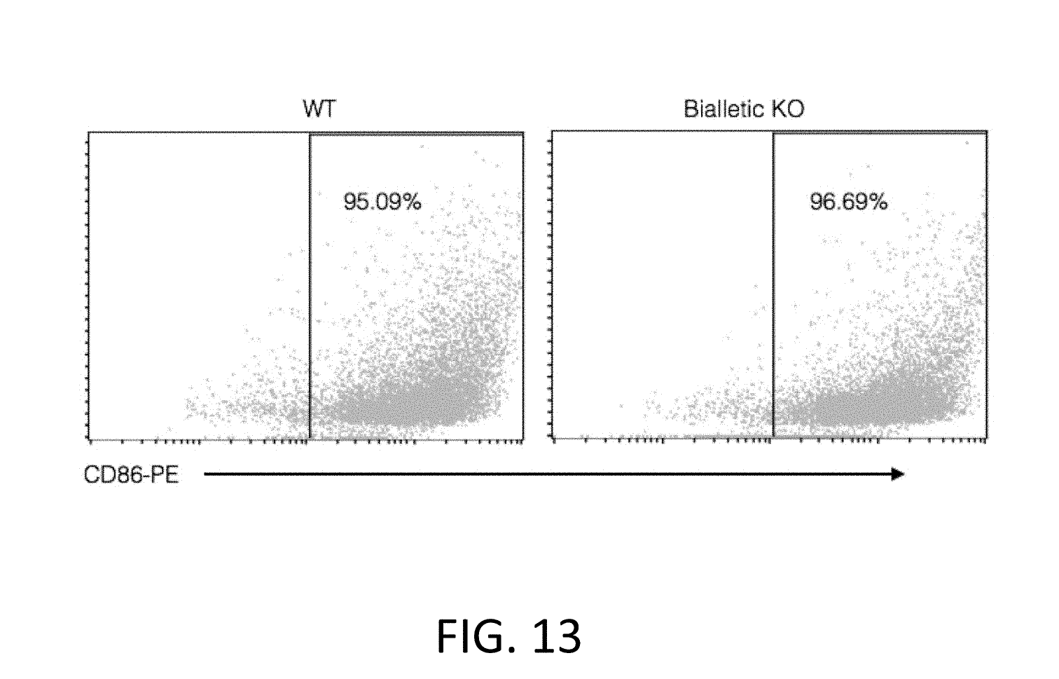

CD86+fibroblasts derived from human pluripotent stem cell were obtained. Human CD86 gene was cloned, and the Lv vectors were constructed with CD86, Lv-CD86-Pure. The CD86 lentivirus was then packaged. Fibroblasts were transfected by CD86 lentivirus, and the positive cells were enriched by 2 μg/ml puromycin. The cells purity was measured by Flow cytometry (FIG. 13).

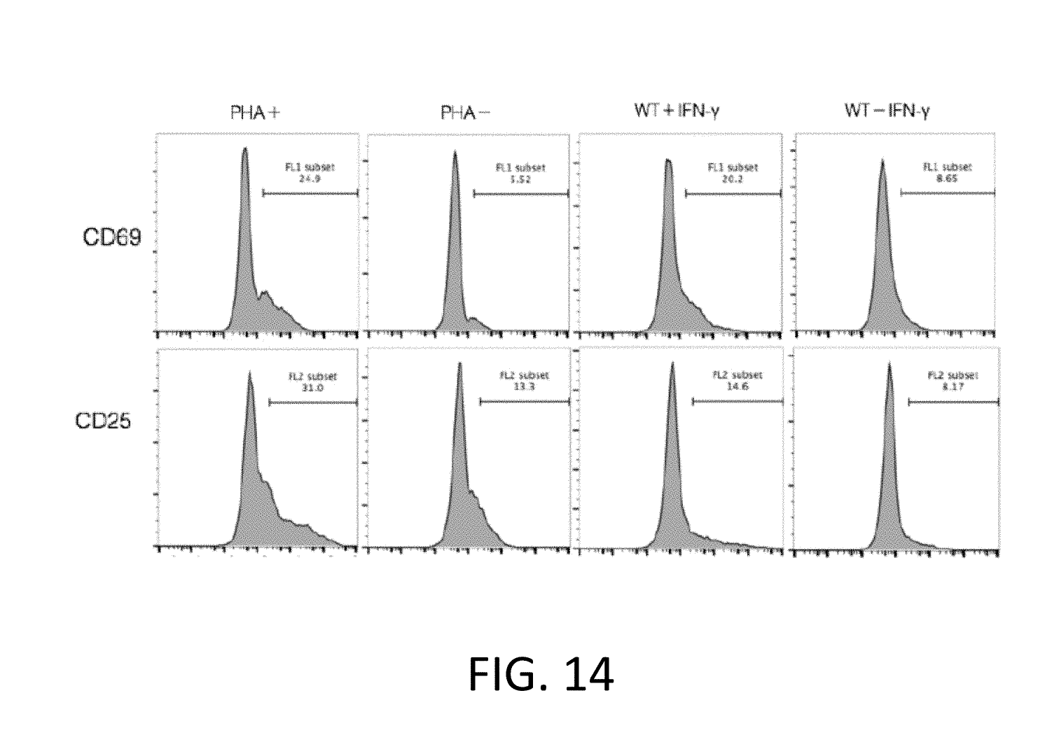

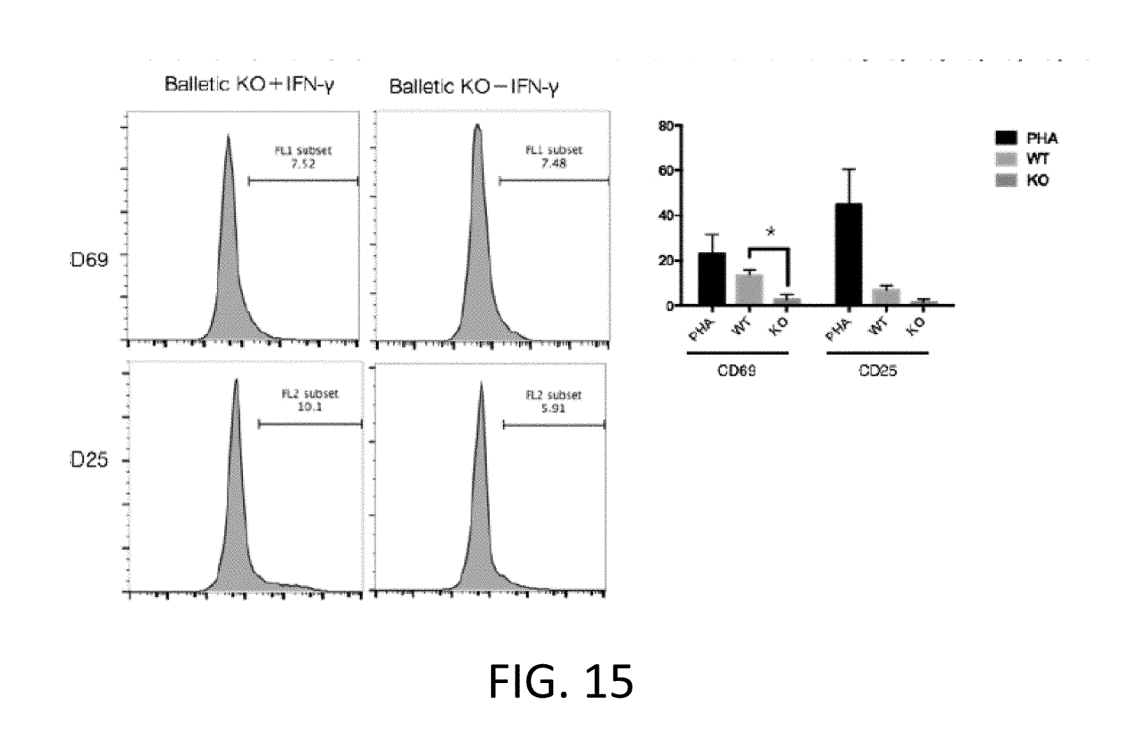

CD4+ T cells stimulation with CD86+fibroblasts were measured. Fibroblasts were seeded in 48 well plates, 2×104 cells per well. The cells were treated with 500 IU/ml IFN-γ for 4 days. The IFN-γ plus medium were removed, and fresh medium was added with 10 μg/ml Mitomycin C. After 2-hour treatment, the Mitomycin C plus medium was removed and washed with PBS twice. 200 μl X-VIVO15 and 2×105 CD4+T cells were added. The positive control was CD4+T cells treated with 2 μg/ml PHA. After 4-day co-culture, T cells were collected to test CD25 and CD69 by flow cytometry (FIGS. 14 and 15).

Fibroblasts express HLA II and stimulate T cells after IFN-γ treatment in vivo. However, it did not happen in vitro for the lack of co-stimulator on fibroblasts such as CD86. The fibroblasts were modified by lentivirus, and the modified CD86+ fibroblasts had constitutive expression of CD86. CD25 and CD69 are the markers of stimulated T cells. CD69 is up-expressed at the initial stage while CD25 is up-expressed at late-stage. The different expression of CD25 and CD69 were checked in CD4+ T cells co-culture with fibroblasts. The medium was animal origin free and contain no IL2, which may stimulate T cells. After 4-day co-culture, CIITA−/− fibroblasts co-culture group had fewer CD69 up-expression. CD25 did not have different expression between CIITA+/+ and CIITA−/− fibroblasts co-culture groups. These results show CIITA knockout decrease the stimulation of CD4+T cells. Further, these results indicate that cells derived from CIITA−/− hESCs may escape from the attack of CD4+ T cells when tissues and/or cells derived from the CIITA−/− hESCs are transplanted into human body.

What is claimed is:

1. A modified cell comprising a disruption of MHC class II transactivator (CIITA) gene, wherein the modified cell is a modified stem cell or a cell derived from the modified stem cell.

2. The modified cell of claim 1, wherein the MHC II is Human Leukocyte antigen II (HLA-II).

3. The modified cell of claim 1, wherein the modified stem cell comprises at least one of a totipotent stem cell, a pluripotent stem cell, an embryonic stem cell, an induced pluripotent stem cell, or a multipotent stem cell.

4. The modified cell of claim 1, wherein the modified stem cell comprises a human embryonic stem cell.

5. The modified cell of claim 1, wherein a karyotype of the modified cell is the same as a karyotype of the corresponding wild-type cell.

6. The modified cell of claim 1, wherein a level of pluripotency of the modified cell is substantially the same as a level of pluripotency of the corresponding wild-type cell.

7. The modified cell of claim 1, wherein the disruption of CIITA gene comprises a disruption of one or more exons of CIITA gene having the nucleic acid sequence of SEQ ID NO: 46.

8. The modified cell of claim 7, wherein the modified cell comprises at least one of the nucleic acid sequence of SEQ ID NOs: 47-58.

9. The modified cell of claim 1, wherein the modified cell has decreased immunogenicity as compared to the corresponding wild-type cell.

10. The modified cell of claim 9, wherein the decreased immunogenicity comprises a decreased level of inflammatory responses induced by the modified cell as compared to the corresponding wild-type cell.

11. A method of reducing T cell responses to presentation of a cell, the method comprising Introducing a disruption of MHC class II transactivator (CIITA) gene of the cell to obtain a modified cell, wherein the cell is a stem cell or a cell derived from the stem cell, the disruption of CIITA of the cell produces a reduced T cell responses to the presentation of the modified cell as compared to a wild-type cell.

12. The method cell of claim 11, wherein the MHC II is HLA-II.

13. The method cell of claim 11, wherein the stem cell comprises at least one of a totipotent stem cell, a pluripotent stem cell, an embryonic stem cell, an induced pluripotent stem cell, or a multipotent stem cell.

14. The method cell of claim 11, wherein the stem cell comprises a human embryonic stem cell.

15. The method cell of claim 11, wherein the T cell is a CD4+ T-cell.

16. The method cell of claim 11, wherein a level of pluripotency of the modified cell is substantially the same as a level of pluripotency of the cell.

17. The method cell of claim 11, wherein the disruption of CIITA gene comprises a disruption of one or more exons of CIITA gene having the nucleic acid sequence of SEQ ID NO: 46.

18. The method cell of claim 17, wherein the modified cell comprises one of the nucleic acid sequence of SEQ ID NOs: 47-58.


SEQUENCE LISTING (58 sequences)

SEQ ID NO: 1 - DNA, 17 bp, Artificial Sequence
  Synthesis
gctgaccccctgtgcct

SEQ ID NO: 2 - DNA, 16 bp, Artificial Sequence
  Synthesis
gaccccctgtgcctct

SEQ ID NO: 3 - DNA, 18 bp, Artificial Sequence
  Synthesis
ctccagccaggtccatct

SEQ ID NO: 4 - DNA, 17 bp, Artificial Sequence
  Synthesis
tctccagccaggtccat

SEQ ID NO: 5 - DNA, 15 bp, Artificial Sequence
  Synthesis
tcagcaggctgttgt

SEQ ID NO: 6 - DNA, 17 bp, Artificial Sequence
  Synthesis
tcagcaggctgttgtgt

SEQ ID NO: 7 - DNA, 15 bp, Artificial Sequence
  Synthesis
ccctggtctcttcat

SEQ ID NO: 8 - DNA, 16 bp, Artificial Sequence
  Synthesis
aagcctccctggtctt

SEQ ID NO: 9 - DNA, 15 bp, Artificial Sequence
  Synthesis
aagcctccctggtct

SEQ ID NO: 10 - DNA, 21 bp, Artificial Sequence
  Synthesis
tacacggtctcctgggtcaag

SEQ ID NO: 11 - DNA, 22 bp, Artificial Sequence
  Synthesis
gtctcttctttacgctgtgcag

SEQ ID NO: 12 - DNA, 24 bp, Artificial Sequence
  Synthesis
tctcaagataatgtcacagaactg

SEQ ID NO: 13 - DNA, 22 bp, Artificial Sequence
  Synthesis
tctgttcactctcttccctctc

SEQ ID NO: 14 - DNA, 22 bp, Artificial Sequence
  Synthesis
aatgtcgtgcaccgattgttcc

SEQ ID NO: 15 - DNA, 21 bp, Artificial Sequence
  Synthesis
actgcttgtggtctccgtacc

SEQ ID NO: 16 - DNA, 22 bp, Artificial Sequence
  Synthesis
gccgagttctatctgaatcctg

SEQ ID NO: 17 - DNA, 22 bp, Artificial Sequence
  Synthesis
cgttctgctgcattgcttttgc

SEQ ID NO: 18 - DNA, 23 bp, Artificial Sequence
  Synthesis
tatcagagctgtgatcttgagag

SEQ ID NO: 19 - DNA, 21 bp, Artificial Sequence
  Synthesis
atcgttggtggcctgagtgtg

SEQ ID NO: 20 - DNA, 21 bp, Artificial Sequence
  Synthesis
acgctacaactctaccgctgc

SEQ ID NO: 21 - DNA, 21 bp, Artificial Sequence
  Synthesis
atgatgaagacagtgcccacc

SEQ ID NO: 22 - DNA, 21 bp, Artificial Sequence
  Synthesis
tgcacctgctccagaatgctg

SEQ ID NO: 23 - DNA, 21 bp, Artificial Sequence
  Synthesis
ccagttccagtgactctttcg

SEQ ID NO: 24 - DNA, 21 bp, Artificial Sequence
  Synthesis
gctgggattcctacacaatgc

SEQ ID NO: 25 - DNA, 22 bp, Artificial Sequence
  Synthesis
gggttctgagtagagctcaatc

SEQ ID NO: 26 - DNA, 25 bp, Artificial Sequence
  Synthesis
tctcacagcttgtaaagcctgagac

SEQ ID NO: 27 - DNA, 24 bp, Artificial Sequence
  Synthesis
aggatggtttcgatctcctgacct

SEQ ID NO: 28 - DNA, 24 bp, Artificial Sequence
  Synthesis
atgtgtctgggtttcatccatccg

SEQ ID NO: 29 - DNA, 24 bp, Artificial Sequence
  Synthesis
tcttcaaacctccatgatgctgct

SEQ ID NO: 30 - DNA, 21 bp, Artificial Sequence
  Synthesis
aagaacacccgcaccaacgag

SEQ ID NO: 31 - DNA, 21 bp, Artificial Sequence
  Synthesis
ctcctggatttcctcttcgtg

SEQ ID NO: 32 - DNA, 28 bp, Artificial Sequence
  Synthesis
gaatgctgcaaactgaccacgctggaac

SEQ ID NO: 33 - DNA, 28 bp, Artificial Sequence
  Synthesis
tggcattcaagagggttttcagtctgga

SEQ ID NO: 34 - DNA, 24 bp, Artificial Sequence
  Synthesis
tcctctttgtcatcacgcttccct

SEQ ID NO: 35 - DNA, 23 bp, Artificial Sequence
  Synthesis
tccagacgccaacatagaccacc

SEQ ID NO: 36 - DNA, 26 bp, Artificial Sequence
  Synthesis
gccctctccctcccctccacgcacag

SEQ ID NO: 37 - DNA, 26 bp, Artificial Sequence
  Synthesis
cggcgccgttgctcacagaccacagg

SEQ ID NO: 38 - DNA, 20 bp, Artificial Sequence
  Synthesis
tgcctgagaaagagaaccag

SEQ ID NO: 39 - DNA, 20 bp, Artificial Sequence
  Synthesis
atggcaggatgaacaaacac

SEQ ID NO: 40 - DNA, 24 bp, Artificial Sequence
  Synthesis
taccaaccaattccacaacccacc

SEQ ID NO: 41 - DNA, 23 bp, Artificial Sequence
  Synthesis
atcataactccgcccattcaccg

SEQ ID NO: 42 - DNA, 27 bp, Artificial Sequence
  Synthesis
cctggaagggctgaccgacgagatcaa

SEQ ID NO: 43 - DNA, 25 bp, Artificial Sequence
  Synthesis
cttcccagccaggctctgcagctcc

SEQ ID NO: 44 - DNA, 22 bp, Artificial Sequence
  Synthesis
aggtcggagtcaacggatttgg

SEQ ID NO: 45 - DNA, 24 bp, Artificial Sequence
  Synthesis
aggctgttgtcatacttctcatgg

SEQ ID NO: 46 - DNA, 57 bp, Homo sapiens
atgctgaccccctgtgcctctaccacttctatgaccagatggacctggctggagaag

SEQ ID NO: 47 - DNA, 32 bp, Artificial Sequence
  Synthesis
atgctgaccccctgtggacctggctggagaag

SEQ ID NO: 48 - DNA, 48 bp, Artificial Sequence
  Synthesis
atgctgaccccctgtgcctctaccaccagatggacctggctggagaag

SEQ ID NO: 49 - DNA, 61 bp, Artificial Sequence
  Synthesis
atgctgaccccctgtgcctctaccacttctatctatgaccagatggacctggctggagaag

SEQ ID NO: 50 - DNA, 80 bp, Artificial Sequence
  Synthesis
atgctgtttactcactccggaacaagtggtcgcaatcgcctccaacacagatggaccacagatggacctggctggagaag

SEQ ID NO: 51 - DNA, 36 bp, Artificial Sequence
  Synthesis
atgctgaccccctgtgcctctacctggctggagaag

SEQ ID NO: 52 - DNA, 28 bp, Artificial Sequence
  Synthesis
atgctgaccccggacctggctggagaag

SEQ ID NO: 53 - DNA, 31 bp, Artificial Sequence
  Synthesis
atgctgacccagatggacctggctggagaag

SEQ ID NO: 54 - DNA, 45 bp, Artificial Sequence
  Synthesis
atgctgaccccctgtgcctctaccagatggacctggctggagaag

SEQ ID NO: 55 - DNA, 49 bp, Artificial Sequence
  Synthesis
atgctgaccccctgtgcctctaatgaccagatggacctggctggagaag

SEQ ID NO: 56 - DNA, 50 bp, Artificial Sequence
  Synthesis
atgctgaccccctgtgcctctctatgaccagatggacctggctggagaag

SEQ ID NO: 57 - DNA, 45 bp, Artificial Sequence
  Synthesis
atgctgaccccctgtgcctctaccacatggacctggctggagaag

SEQ ID NO: 58 - DNA, 58 bp, Artificial Sequence
  Synthesis
atgctgaccccctgtgcctctaccactctatatgaccagatggacctggctggagaag